(19) United States
(12) Patent Application Publication
(10) Pub. No.: US 2019/0111173 A1
(43) Pub. Date: Apr. 18, 2019

(54) Fragrance-Emitting Item

(71) Applicant: SAS CARESTIA, Mouans Sartoux (FR)

(72) Inventor: Marie-Hélène MARCELLI, VILLENEUVE LOUBET (FR)

(21) Appl. No.: 16/162,606

(22) Filed: Oct. 17, 2018

(30) Foreign Application Priority Data
Oct. 17, 2017 (FR) 1759738

(51) Int. Cl. A61L 9/12

(52) U.S. Cl. CPC A61L 9/125; B32B 7/06

(57) ABSTRACT

Disclosed is a containment component and a fragrant multi-layer assembly unit which may include at least three layers, namely a sheet forming a self-supporting carrier of the fragrance-emitting item, an adhesive interface and a liner, the sheet, which extends surface wise with a peripheral boundary, is made of paper, has a certain thickness or surface permeability on the front side, is adapted and designed to be impregnated therein with a fragrant fluid, includes a front forming the external face visible to the user of the fragrant multi-layer assembly unit, the fragrant fluid is received by the sheet, which in itself is a fragrance-emitting sheet, the scent of the fragrant fluid is released automatically and gradually from the fragrance-emitting sheet when the latter is placed in the ambient atmosphere.

### BACKGROUND OF THE INVENTION

### Field of the Invention

The invention relates to a decorative, informative or promotional fragrance-emitting item, distributed at the point of sale or as an insertion in newspapers, periodicals, bulk mailings, interactive distribution terminals, to customers or potential buyers, by also transmitting to them an olfactory universe, which is adapted and intended, when in use, to be attached to a support so as to showcase a decorative, informative or promotional surface and give off the fragrance to the surrounding atmosphere ephemerally.

### Description of the Related Art

GB 2 174 526 discloses a V-shaped fragrance sales tool and information item which is foldable or open, made of a sheet of paper provided with folds, each supporting a sample fragrance in a protected form but releasable by scratching. The item forms “scratch and sniff” fragrance test panels.

FR 2 729 681, FR 2 943 581, EP 0 734 713 and EP 1 075 964 describe microcapsules of an odorant borne by a support. The microcapsules are broken in order to release the odorant.

EP 1 243 199 discloses a fragrance test sample which comprises a case with a frangible opening portion, sealed prior to use, and a support element made of absorbent material impregnated with perfume in fluid state, disposed in the case.

U.S. Pat. No. 4,283,011 discloses a fragrance-emitting item for a garment.

US 2005/0196571 discloses an air freshener being a closed envelope in which is placed a assembly unit including a non-adhesive silicone-coated dorsal element, a vinyl sheet which, on the front side, is covered with a coat of paint or ink which has been mixed with a fragrant fluid and which, on the inner side, has an adhesive layer associated with the non-adhesive dorsal element. Such an air freshener is attached to a window and shows the decorative surface consisting of the coat of paint or ink.

US2007/014959 relates to a “toilet lid cover appliqué forming a unit” which comprises, in the initial state, a support back panel, a release layer, an outer decorative layer having an appliqué with figurative art elements and intended to be fixed on the toilet lid, a picture frame portion meant for forming a clevis, and a cutting line between the appliqué and the picture frame portion. Three embodiments are envisaged. In the first embodiment, the support back panel is made of paper, the release layer is applied to the front face of the back panel and the decorative layer is a thin film of plastic material that can adhere statically without requiring adhesive. In the second embodiment, the support back panel is made of plastic, the front side of the back panel acts as a release layer, the decorative layer is a thin film of plastic material and the adhesion is by static electricity. In the third embodiment, the support back panel is made of paper, the release layer is applied to the front side of the back panel, and there is a release layer which is distinct from the back panel, the decorative layer is made of paper and adheres to the release layer by means of an adhesive, and adheres to the surface on which it is applied thanks to the adhesive on the reverse side. The “appliqué” is not intended to be placed in a containment component that would be open only for using the appliqué. On the contrary, there is provision on the picture frame portion for the presence of the clevis which forms a through hole

DE 203 10 677 discloses a sticker for self-adhesive skin. The base stock may be PVC. The release paper is perforated for easy removal. The adhesive is a polyacrylate mass applied in dots.

DE 20 2008 005251 discloses a fragrance dispenser with fragrance support, such as fragrance-impregnated cardboard, and a plastic film package, particularly for the interior of vehicles.

The problem underlying the invention is to have a decorative, informative or promotional fragrance-emitting item, distributed at the point of sale or as an insertion in newspapers, periodicals, bulk mailings, interactive distribution terminals, to customers or potential buyers, by also transmitting to them an olfactory universe, which is adapted and intended, when in use, to be attached to a support so as to show the user a decorative, informative or promotional surface and give off the fragrance to the surrounding atmosphere ephemerally, automatically and gradually without requiring any specific action for this purpose such as scraping, rubbing or breaking the fragrance microcapsules. It is a question of having such an item which can, (i) comprise at least three layers, (ii) be easily fixed, without preparation or damage to any resistant carrier having a point-to-point, linear or surface-wise fastening area, (iii) be removed from the support to which it was previously fastened just as easily, without damage, and (iv) be repositioned if possible.

### SUMMARY OF THE INVENTION

A disclosure of the invention as characterized shall be explored below.

The invention relates to such a fragrance-emitting item, in the state before use, adapted and intended in the state of use to be attached to a support so as to show the user a decorative, informative or promotional surface and give off the fragrance to the surrounding atmosphere ephemerally, combining a fragrant multi-layer assembly unit with a fragrant fluid incorporated in one layer, an adhesive interface being integral with the back of the sheet, and a liner detachably associated to the adhesive interface, and a containment component of the assembly unit so as to prevent the scent of the fragrant fluid from perfuming the ambient atmosphere before the first use, said containment component being adapted and intended to be opened, such that, when in use, the assembly unit having been extracted from the containment component and the liner having been separated from the adhesive interface, the adhesive interface can be glued to the support.

This fragrance-emitting item is such that:
the assembly unit may comprise at least three layers, namely a sheet forming a self-supporting carrier for the fragrance-emitting item, the adhesive interface and the liner;
said sheet, which extends surface-wise with a peripheral boundary:
   is made of paper;
   has a certain porosity;
   is adapted and intended to be the vector of the scent of a fragrant fluid;
   has a back and a front forming the outer face visible to the user of the assembly unit;
the fragrant fluid—a typically alcoholic solution made from a concentrate—is incorporated either in said sheet or in the adhesive interface, said sheet being a fragrance-emitting sheet;
the adhesive interface is of the type used for a self-adhesive item and is on the back of the fragrance-emitting sheet;
the liner is of the type used to protect an adhesive interface of a self-adhesive item prior to use and having been separated from the adhesive interface, the adhesive interface can be glued to the support and the perfumed multilayer assembly unit fixed to the support,
the fragrance-emitting item has an overall thickness of the order of magnitude of a millimeter and an overall length and width of the order of magnitude of a centimeter or a few centimeters,

the scent of the fragrant fluid is released automatically and gradually from the fragrance-emitting sheet when the latter is placed in the ambient atmosphere, so as to give off the fragrance of the olfactory universe ephemerally.

According to the first form of embodiment, the fragrant fluid is incorporated in the adhesive interface, the scent of the fragrant fluid being released from the fragrance-emitting sheet in contact with the adhesive interface such that the scent of the fragrant fluid migrates from the adhesive interface up to the fragrance-emitting sheet.

According to a first form of embodiment, the fragrant fluid is incorporated into the fragrance-emitting sheet by impregnation the latter.

According to one embodiment, towards its back side the fragrance-emitting item also has a substrate which extends surface-wise with a peripheral boundary, having a back and a front of which is integral with the liner which is invisible to the user.

As the case may be, the peripheral boundary of the substrate and the peripheral boundary of the fragrance-emitting sheet coincide substantially or the peripheral boundary of the substrate goes beyond the peripheral boundary of the fragrance-emitting sheet, the peripheral boundary of the fragrance-emitting sheet going below the peripheral boundary of the substrate, the item comprising a framing sheet having an inner boundary and an outer boundary, the framing sheet being disposed between the peripheral boundary of the substrate and the peripheral boundary of the fragrance sheet.

According to one possibility, the inner limit and the outer limit of the framing sheet and, respectively, the peripheral boundary of the fragrance-emitting sheet and the peripheral boundary of the substrate substantially coincide.

According to one possibility, the fragrance-emitting sheet and the framing sheet are parts of the same sheet, the inner limit of the framing sheet and the peripheral boundary of the fragrance-emitting sheet being formed by one and the same cutting line of the same leaf.

As the case may be, the fragrance-emitting item comprises either a single fragrance-emitting sheet or several fragrance-emitting sheets that are spaced apart from each other or at least partially adjoining.

As the case may be, and alternatively or cumulatively:
the front of the fragrance-emitting sheet, where appropriate the front of the framing sheet, comprises visible decorative, informative or promotional printings;
the fragrance-emitting sheet has a basis weight of between approximately 200 and 300 g/m²;
the liner is a silicone glassine paper on one side;
the adhesive interface is removable or repositionable.

According to one characteristic, the containment component of the fragrant multi-layer assembly unit is fitted the latter, so as to confine the scent of the fragrant fluid.

### BRIEF DESCRIPTION OF THE DRAWINGS

Here is a brief description of the figures of the drawings.

FIG. 1 is a schematic view from the front of a fragrance-emitting item according to one of two forms of embodiment, as is prior to use, and according to a first embodiment in which the fragrance-emitting item comprises a single fragrance-emitting sheet, showing the fragrant multi-layer assembly unit, namely the front of the fragrance-emitting sheet and the containment component of this assembly, 1 herein transparent.

FIG. 2 is a schematic cross-sectional view of the item along the line 2-2 of FIG. 1, showing the fragrant multi-layer assembly unit with three layers, namely from the front to the rear, the fragrance-emitting sheet, the adhesive interface being integral with the back of the fragrance-emitting sheet, and the liner.

FIG. 3 is a schematic cross-sectional view of the item of FIG. 1, when in use, where the fragrant multi-layer assembly unit extracted from the containment component is fixed to a support so that the scent of the fragrant fluid is released automatically and gradually from the fragrance-emitting sheet placed in the ambient atmosphere.

FIG. 4 is a schematic cross-sectional view of the item similar to FIG. 1, according to a second embodiment, in which the fragrant multi-layer assembly unit comprises four layers, namely from the front to the rear, the fragrance-emitting sheet, the adhesive interface being integral with the back of the fragrance-emitting sheet, the liner and a substrate having a back and a front, which is integral with the liner, the peripheral boundary of the substrate and the peripheral boundary of the fragrance-emitting sheet coinciding substantially.

FIG. 5 is an exploded schematic sectional view of the fragrant multi-layer assembly unit of FIG. 4, on a larger scale.

FIG. 6 is a schematic view from the front, similar to FIG. 1, of an item in the state before use according to a third embodiment in which the item comprises several spaced out fragrance-emitting sheets, the peripheral boundary of a fragrance-emitting sheet going below the peripheral boundary of the substrate, the item comprising a framing sheet having an inner limit and an outer limit, wherein the inner boundary and the outer boundary of the framing sheet and, respectively, the peripheral boundary of the fragrance-emitting sheet and the peripheral boundary of the substrate substantially coincide, the fragrance-emitting sheet and the framing sheet being parts of the same sheet, the inner limit of the framing sheet and the peripheral boundary of the fragrance-emitting sheet being formed by the same cutting line of the same sheet.

FIG. 7 is a schematic cross-sectional view of the item taken along the line 7-7 of FIG. 6 showing the fragrant multi-layer assembly unit with four layers, and the framing sheet, the fragrance-emitting sheet as well as the framing sheet being the parts of the same sheet, the inner limit of the framing sheet and the peripheral boundary of the fragrance-emitting sheet being formed by a same cutting line of the same sheet.

### DESCRIPTION OF THE PREFERRED EMBODIMENTS

In the figures, in order that the figures be easier to read, the different constituent parts are not represented on the same scale. Thus, any thin layer is shown with a greater relative thickness.

A disclosure of the several forms of embodiment shall be explored below with examples.

Item 1, decorative, informative or promotional, is meant to be distributed at the point of sale or as an insertion in newspapers, periodicals, bulk mailings, interactive distribution terminal, to customers or potential buyers, by also transmitting to them an olfactory universe. It is adapted and intended, when in use, to be attached to a support so as to show the user a decorative, informative or promotional surface and give off the fragrance to the surrounding atmosphere ephemerally, automatically and gradually without requiring any specific action for this purpose such as scraping, rubbing or breaking the fragrance microcapsules. It can be designed and distributed by a company to promote its brand, its product or its service to customers or potential buyers, by transmitting them by the same token an olfactory universe. It can accompany these customers or potential buyers in their own personal or professional universe and can be easily fixed, without preparation or damage to any resistant support S having a point-to-point, linear or surface-wise fastening area, be removed from the support in question just as easily and without damage, be repositioned if possible, and give off the fragrance, ephemeral, that is to say, not particularly long lasting as would be the case for an air freshening item for example. The amount of fragrance in item 1 may be limited, as in a sample, since item 1 is not designed to be refilled with fragrance, but may remain attached to the support even after having given of the fragrance, given its decorative aspect.

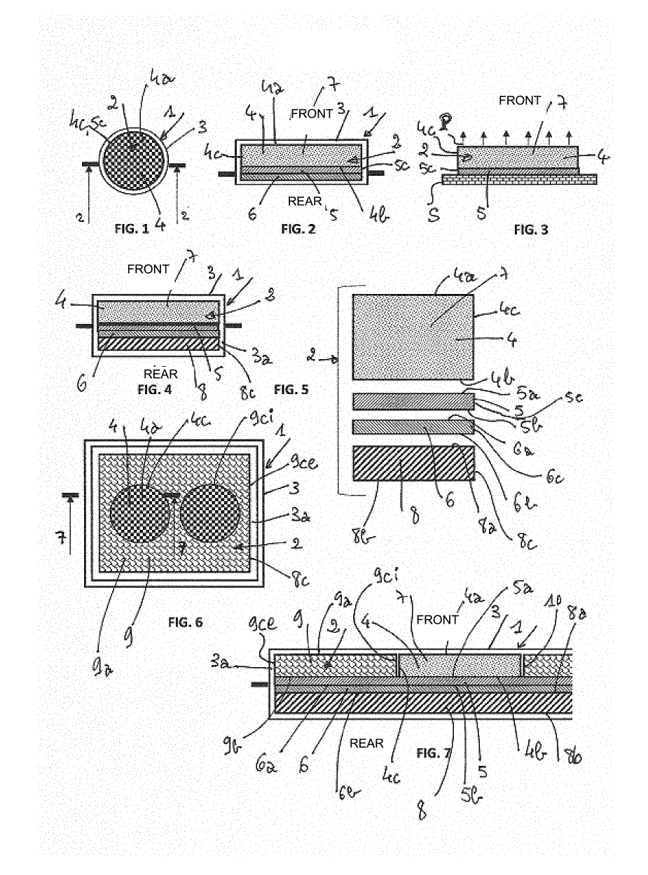

Item 1 is in two possible states: the state before use (FIGS. 1, 2, 4, 6 and 7) and the state of use (FIG. 3) in which it is adapted and intended to be fixed to the support S by showing a decorative, informative or promotional surface and give off the fragrance into the surrounding atmosphere (arrows P, FIG. 3).

Support S is strong enough to receive and carry item 1. It has a point-to-point, linear or surface-wise fastening area. It can be flat or not, plane or not. It can be rigid or deformable with a certain degree, for example curved, item 1 having been made deformable and adaptable to the shape of the support S. Such a support S may be a wall, an inner surface, a piece of furniture, an object.

In the state before use, item 1 combines a fragrant multi-layer assembly unit 2 and an envelope 3 of the assembly unit 2.

The envelope 3 limits an interior space 3a having a significant degree of tightness, in which the assembly unit 2 is placed, by preventing the fragrance from escaping from the assembly unit 2 and perfuming the ambient atmosphere before the first use of the item, while this is not desired. The envelope 3 is adapted and intended to be opened and the assembly unit 2 can then be extracted therefrom.

In one embodiment, the envelope 3 is fitted to the assembly unit 2, so as to confine the fragrance of item 1. The space 3a then has dimensions corresponding to those of the assembly unit 2, so as to leave a limited or minimal space between them, for the necessary clearances in order to insert and pull out the assembly unit 2.

According to the embodiments, the opening of the envelope 3 is final once done, or the envelope 3 is adapted and intended to be closed in on itself.

In the embodiment shown in the figures, the envelope 3 is completely transparent so that the user can see through the assembly unit 2 placed in the space 3a. In another embodiment, the envelope 3 is only partially transparent, inasmuch as it is provided for example with a transparent window. In another embodiment, the envelope 3 is opaque.

In one embodiment, the envelope 3 is made of paper or is paper-based.

In one embodiment, the flat envelope 3 forms a kind of flat and thin bag.

In the embodiment shown in the figures, the envelope 3 is completely distinct from the assembly unit 2 from which it is completely separated in view of the state of use. In another embodiment, the envelope 3 is associated with the physically fragrant multi-layer assembly unit 2, for example by being fixed to it, including the state of use, while it has been opened.

One of two forms of embodiments is described according to several embodiments.

According to a first form of embodiment (FIGS. 1 to 3), the assembly unit 2 comprises a minimum number of contiguous successive layers, i.e., three layers, namely from the front AV to the rear AR, a single sheet 4, an adhesive interface 5 and a liner 6. The assembly unit 2 also includes a fragrant fluid 7.

The term “successive” applied to several layers must be understood as meaning that these layers follow each other in a certain order. The term “contiguous” applied to two successive layers must be understood as meaning that these layers touch one another to be in contact.

The term “layer” means a material in a more or less uniform extended form with a small thickness relative to the surface area. Thus, a layer may be referred to as “sheet” or “sheet”, “interface”, “strip”, “coating”, “liner” (in the sense of layer protecting a sticky side). The term “substrate” should also be understood as a “layer”. Layers are differentiated from each other by the constituent material, thickness and other properties. Thus, the terms “sheet” and “substrate” are generally used to denote layers of greater relative thickness, while the terms “interface”, “film”, “coating”, “liner” are generally used to denote layers of smaller relative thickness.

The term “front” AV should be understood as what is facing the direction of the user placed in front of item 1 being in the relative position corresponding to that in which the assembly unit 2 is fixed to the support S. The term “back” AR, should be understood as what is turned away from the front AV.

The sheet 4 has a front 4a and a back 4b. It extends surface-wise and has a peripheral boundary 4c. The front 4a of the sheet 4 is free and intended to be visible by the user forming the outer face visible to the user of the assembly unit 2. The term “free” should be understood as not covered with a layer. The back 4b is invisible.

The term “front” and “back” of a layer of the assembly unit 2, should be understood, respectively, as the front and the back of this layer, namely the two faces thereof which are turned respectively towards the front AV and the rear AR. Thus defined, the terms “front” and “back”, taken as such without further specification, are independent, respectively, of the characteristics “intended to be seen” (or otherwise expressed “visible”) and “intended not to be seen” (or otherwise expressed as “invisible”) in relation to the user.

All the faces of the layers of the assembly unit 2 respectively facing forward AV and rearward AR are respectively called front and back.

The shape of the peripheral boundary 4c is depends on decorative, informational or promotional needs. For example, the peripheral boundary 4c is either a simple shape (such as a circle—FIGS. 1 and 6), a polygon, a figurine (like an animal, a fruit), a logo, the shape of a product or the symbolizing shape a service, such as the one which item 1 promotes.

In one embodiment, the front 4a, free, of the sheet 4 may comprise visible printings with a decorative, informative or promotional function, having no function with regard to the fragrant fluid 7. For example, the front 4a of the sheet 4 can be embossed, so as to create relief blocks.

The sheet 4 is not a plastic sheet (as a vinyl sheet would be). Indeed, according to the invention, the sheet 4 is made of paper (which includes the case where it would be essentially paper-based), as a blotting paper. It has a basis weight of ranging between 200 g/m² and 300 g/m², which corresponds to paper that can be described as thick.

The sheet 4 has a certain thickness or surface permeability on the front side 4a.

In one embodiment, the sheet 4 is pure cellulose fibers, with very little or without glue, with minimum mineral fillers, at neutral pH.

The function of sheet 4 is, on the one hand, to be a self-supporting carrier of item 1, and, on the other hand, to serve as a support for the fragrant fluid 7 incorporated within sheet 4 itself. Sheet 4 is chosen as it is adapted and designed to be impregnated with all of the fragrant fluid 7 of item 1, since the fragrant fluid 7 is not located elsewhere but within sheet 4 (particularly since it is not in a separate layer on a sheet which is therefore not fragrance-emitting). Thus, the sheet 4, per se, is a fragrance-emitting sheet.

According to the first and second forms of embodiment (FIGS. 1, 2, 4 and 5), the assembly unit 2, and therefore item 1, comprises a single fragrance-emitting sheet 4 consisting of a single fragrance-emitting unit.

Alternatively, the assembly unit 2, and therefore item 1, comprises a single fragrance-emitting sheet 4 consisting of several fragrance-emitting units arranged side by side so as to constitute the single fragrance-emitting sheet 4.

The adhesive interface 5 has a front 5a and a back 5b, extends surface-wise, and has a peripheral boundary 5c. The front 5a of the adhesive interface 5 is integral with the back 4b of the fragrance-emitting sheet 4.

The liner 6 has a front 6a and a back 6b, extends surface-wise, and has a peripheral boundary 6c. The front 6a of the liner 6 covers the back 5b of the adhesive interface 5.

According to the first form of embodiment (FIGS. 1 and 2), the back side 6b of the liner 6 is free.

According to the first and second forms of embodiment (FIGS. 1, 2, 4 and 5), the peripheral boundaries 4c, 5c and 6c of the fragrance sheet 4, the adhesive interface 5 and the liner 6 coincide, being located directly above each other. Where appropriate, the liner 6 has a protruding gripping portion to be gripped for removal.

In the state of use (FIG. 3), the assembly unit 2 has been previously released from the envelope 3 and the liner 6 has been separated from the adhesive interface 5. Thus, the adhesive interface 5 can be glued on the support S and the assembly unit 2 fixed to the support S. The scent of the fragrant fluid 7 which has been captured by the fragrance-emitting sheet 4 is then released automatically and gradually from the fragrance-emitting sheet 4 when the latter is placed in the atmosphere ambient, without needing any action like scratching for example.

The adhesive interface 5 which is on the back 4b of the fragrance-emitting sheet 4 must be compatible with the fragrant fluid 7 which impregnates the fragrance-emitting sheet 4 therein. It is typically of the type used for self-adhesive items.

According to the embodiments, the adhesive interface 5 is removable or repositionable. The term “removable” should be understood as meaning that the adhesive interface 5 has a low final bond on the support S, so that it can be removed easily and without damage to the support S. The term “repositionable” should be understood as meaning that the adhesive interface 5 after being removed from a support S can be repositioned on the same support S at the same place or at a different place or repositioned on another support and with similar or the same bonding qualities.

The liner 6 may typically be a layer of the type used to protect an adhesive interface of a self-adhesive item prior to use.

According to one embodiment, the liner 6 is a one-sided silicone glassine paper, such as a paper with a basis weight of the order of 80 g/m².

The fragrant fluid 7 is a typically alcoholic solution made from a concentrate. It is designed to give off the fragrance of the solution when placed in the atmosphere. This solution is deposited on the sheet 4 so as to impregnate it. This deposit can be achieved by implementing the techniques of the printing field. Thus the scent of the fragrant fluid 7, on the one hand, is captured by the fragrance-emitting sheet 4 and remains captured by it, as long as the assembly unit 2 is confined in the envelope 3. And so the scent of the fragrant fluid 7, on the other hand, is then released automatically and gradually from the fragrance-emitting sheet 4 when the latter is placed in the ambient atmosphere, without requiring any action such as scraping, for example, once that the assembly unit 2 has been pulled out from the envelope 3 to be placed in the ambient atmosphere and fixed to the support S, after removal of the liner 6.

Item 1 is intended to give off the fragrance in ephemerally. It should be understood that Item 1 is for decorative purposes, informative or promotional is not intended to give off the fragrance in a sustainable manner over a long period of time as is the case for example for a freshener of air, for which the durability of exhalation is sought. As a result, the amount of scented fluid 7 of item 1 can be limited, like a fragrance sample, or a little more. Furthermore, item 1 is not designed or intended to be refilled with fragrance.

Referring now to FIGS. 4 and 5 which illustrate the second form of embodiment of item 1, wherein the assembly unit 2 comprises four layers, namely from the front AV to the rear AR and in addition to the fragrance-emitting sheet 4, the adhesive interface 5 and the liner 6 already considered in the first form of embodiment, a substrate 8, located on the back of the item 1, opposite the fragrance-emitting sheet 4.

The substrate 8 has a front 8a and a back 8b, extends surface-wise, and has a peripheral boundary 8c. The back 7b of the liner 7 is integral with the front 8a of the substrate 8, which is invisible. The back 8b of the substrate 8 is free and intended to be visible to the user as soon as he returns the assembly unit 2. The peripheral boundaries 4c, 5c and 6c of the fragrance-emitting sheet 4, the adhesive interface 5 and liner 6 substantially coincide with the peripheral boundary 8c of the substrate 8.

The second embodiment provides the advantage of masking the liner 7 from the user's view and taking advantage of its back 8b, free, to provide visible printings with a decorative, informative or promotional function.

Reference is now made to FIGS. 6 and 7 which illustrate the third form of embodiment, which, like the second form of embodiment of FIGS. 4 and 5, comprises a substrate 8. The peripheral limit 8c of the substrate 8 then goes beyond the peripheral limit 4c of the fragrance-emitting sheet 4, or, which amounts to the same, the peripheral limit 4c of the fragrance-emitting sheet 4 goes below the peripheral limit 8c of the substrate 8. The item 1 also comprises a framing sheet 9 disposed between the peripheral boundary 8c of the substrate 8 and the peripheral boundary 4c of the fragrance-emitting sheet 4. In addition, it is possible for the item 1 to comprise several fragrant sheets 4 distinct from each other (two fragrance-emitting sheet 4 in FIG. 6).

As the case may be, the various fragrance-emitting sheet 4 are spaced out from each other with a space between them, as in the embodiment shown in FIG. 6, or they are adjacent, at least partly. As the case may be, the various fragrance-emitting sheets 4 are identical in shape and size, as in the embodiment shown in FIG. 6, or their shapes and/or their dimensions are different.

In a particular embodiment, the various fragrance-emitting sheets 4 capture the same fragrant fluid 7.

The framing sheet 9 has a front 9a and a back 9b, extends surface-wise, and has an outer peripheral boundary 9ce and an inner peripheral boundary 9ci. The front 9a of the framing sheet 9 is free and intended to be visible to the user, forming the outer face visible to the user of the assembly unit 2. The back 9b is invisible.

In one embodiment, the front 9a, free, of the sheet 4 may comprise visible printings with a decorative, informative or promotional function.

In the embodiment shown in FIGS. 6 and 7, the inner limit 9ci and the outer limit 9ce of the framing sheet 9 and, respectively, the peripheral limit 4c of the fragrance-emitting sheet 4 and the peripheral boundary 8c of the substrate 8, coincide substantially.

In one embodiment (FIG. 7), the fragrance-emitting sheet 4 and the framing sheet 9 are parts of the same sheet, the inner limit of the framing sheet 9ci and the peripheral boundary 4c of the fragrance-emitting sheet 4 being formed by the same cutting line 10 of the same sheet. The cutout then made is called “semi cut peel”, the liner 7 and the substrate 8 are not cutout.

In the third embodiment with an item 1 comprising several fragrance-emitting sheets 4 distinct from each other, the user pulls out the assembly unit 2 from the envelope 3 and then takes one or more sheets 4 from the substrate 8. If one or more fragrance-emitting sheets 4 remain on the substrate 8, the user encloses the substrate 8 with this or these sheets so as to confine it. If necessary, the envelope 3 may be used if it is reusable. In this form of embodiment, the fragrant fluid 7 is incorporated directly into the fragrance-emitting sheet 4 by impregnation thereof during the manufacturing process.

Let us now describe the other of the two forms of embodiment, compared to the previous one. This form of embodiment differs from the previous one in that the fragrant fluid 7 is not incorporated directly into the fragrance-emitting sheet 4, but is incorporated in the adhesive interface 5, the scent of the fragrant fluid 7 being released from the fragrance-emitting sheet 4 in contact with the adhesive interface 5 so that the scent of the fragrant fluid 7 migrates from the adhesive interface 5 to the fragrance-emitting sheet 4. For example, a fragrance concentrate is incorporated in the adhesive intended for the interface adhesive, 20% to 30%. The various embodiments envisaged for the form of embodiment described above can be transposed to this other form of embodiment. In addition, this form of embodiment differs from the previous one in that it is possible to dispense with alcohol for the fragrant fluid 7. Then follows a more limited evaporation, the minimization of the loss of the top notes of the perfume and a slower diffusion which prolongs the service life of the item.

1. A decorative, informative, promotional fragrance-emitting item (1) distributed at the point of sale or as insertion in newspapers, periodicals, postal bulk mailings, interactive distribution terminals, to customers or potential buyers, by also transmitting to them an olfactory universe, as is before use, adapted and intended when in use to be fixed to a support (S) so as to show the user's view a decorative, informative or promotional surface and to give off the fragrance in the surrounding atmosphere, combining, on the one hand, a scented multilayer unit (2) with a fragrant fluid incorporated in a layer, an adhesive interface (5) forming a body with the back of the scented multilayer assembly (2), and a liner (6) releasably associated with the adhesive interface (5), on the other hand, a containment component (3) of the fragrant multi-layer assembly unit (2) so as to prevent the scent of the fragrant fluid from perfuming the ambient atmosphere before the first use of the fragrance-emitting item (1), said containment component (3) being adapted and intended to be open, such that, when in use, the fragrant multi-layer assembly unit (2) having been pulled out from the containment component (3) and the liner (6) having been separated from the adhesive interface (5), the adhesive interface (5) can be stuck on the support (S), wherein
the fragrant multi-layer assembly unit may comprise at least three layers, namely a sheet forming a self-supporting carrier for the fragrance-emitting item, the adhesive interface and the liner;
said sheet (4), which extends surface-wise with a peripheral boundary (4c):
is made of paper;
has a certain porosity;
is adapted and intended to be the vector of the fragrance of a fragrant fluid (7);
has a back and a front (4a) forming the outer face visible to the user of the unitary assembly (2);
the fragrant fluid (7)—a typically alcoholic solution made from a concentrate—is incorporated either in said sheet (4) or in the adhesive interface (5), said sheet (4) being a fragrance-emitting sheet (4);
the adhesive interface (5) is of the type used for a self-adhesive item and is on the back (4b) of the fragrance-emitting sheet (4);
the liner (6) is of the type used to protect an adhesive interface of a self-adhesive item prior to use and having been separated from the adhesive interface (5), the adhesive interface (5) can be glued to the support (S) and the perfumed multilayer assembly unit (2) fixed to the support (S);
the fragrance-emitting item (1) has an overall thickness of the order of magnitude of a millimeter and an overall length and width of the order of magnitude of a centimeter or a few centimeters,
the scent of the fragrant fluid (7) is released automatically and gradually from the fragrance-emitting sheet (4) when the latter is placed in the ambient atmosphere, so as to give off the fragrance of the olfactory universe ephemerally.

2. The fragrance-emitting item of claim 1, wherein the fragrance-emitting item (1) in the fragrant fluid (7) is incorporated in the adhesive interface (5), the scent of the fragrant fluid (5) being released from the fragrance-emitting sheet (4) in contact with the adhesive interface (5) such that the scent of the fragrant fluid (5) migrates from the adhesive interface (5) up to the fragrance-emitting sheet (4).

3. The fragrance-emitting item of claim 1, wherein the fragrant fluid (7) is incorporated in the fragrance-emitting sheet (4) by impregnation thereof.

4. The fragrance-emitting item of claim 1, further comprising towards its back side, a substrate (8) which extends surface-wise with a peripheral boundary (8c), having a back (8b) and a front (8a) which is integral with the liner (6) which is invisible to the user.

5. The fragrance-emitting item of claim 4, wherein the peripheral boundary (8c) of the substrate (8) and the peripheral boundary (4c) of the fragrance-emitting sheet (4) substantially coincide.

6. The fragrance-emitting item of claim 4, wherein the peripheral boundary (8c) of the substrate (8) goes beyond the peripheral limit (4c) of the fragrance-emitting sheet (4), the peripheral limit (4c) of the fragrance-emitting sheet (4) going below the peripheral boundary (8c) of the substrate (8), the fragrance-emitting item (1) comprising a framing sheet (9) having an inner limit (9ci) and a limit outer (9ce), the framing sheet (9) being disposed between the peripheral boundary (8c) of the substrate (8) and the peripheral boundary (4c) of the fragrance-emitting sheet (4).

7. The fragrance-emitting item of claim 6, wherein the inner limit (9ci) and the outer limit (9ce) of the framing sheet (9) and, respectively, the peripheral boundary (4c) of the fragrance sheet (4) and the peripheral boundary (8c) of the substrate (8) substantially coincide.

8. The fragrance-emitting item of claim 7, wherein the fragrance-emitting sheet (4) and the framing sheet (9) are parts of the same sheet, the inner limit (9ci) of the framing sheet (9) and the peripheral boundary (4c) of the fragrance sheet (4) being formed by the same cutting line (10) of the same sheet.

9. The fragrance-emitting item of claim 1, comprising either a single fragrance-emitting sheet (4) or several fragrance-emitting sheet (4) which are spaced out from each other or are at least partly adjoined.

10. The fragrance-emitting item of claim 1, wherein, both/either:
The front (4a) of the fragrance-emitting sheet (4), where appropriate, the front (9a) of the framing sheet (9), comprises visible decorative, informative or promotional printings;
the fragrance-emitting sheet (4) has a basis weight of between approximately 200 and 300 g/m²;
the liner (6) is a silicone glassine paper on one side;
the adhesive interface (5) is removable or repositionable.

11. The fragrance-emitting item of claim 1, wherein the containment component (3) of the fragrant multi-layer assembly unit (2) is fitted thereto, so as to confine the scent of the fragrant fluid (7).

12. The fragrance-emitting item of claim 2, the fragrance-emitting item (1) which also comprises towards its back side, a substrate (8) which extends surface-wise with a peripheral boundary (8c), having a back (8b) and a front (8a) which is integral with the liner (6) which is invisible to the user.

13. The fragrance-emitting item of claim 3, the fragrance-emitting item (1) which also comprises towards its back side, a substrate (8) which extends surface-wise with a peripheral boundary (8c), having a back (8b) and a front (8a) which is integral with the liner (6) which is invisible to the user.

14. The fragrance-emitting item of claim 2, comprising either a single fragrance-emitting sheet (4) or several fragrance-emitting sheet (4) which are spaced out from each other or are at least partly adjoined.

15. The fragrance-emitting item of claim 3, comprising either a single fragrance-emitting sheet (4) or several fragrance-emitting sheet (4) which are spaced out from each other or are at least partly adjoined.

16. The fragrance-emitting item of claim 4, comprising either a single fragrance-emitting sheet (4) or several fragrance-emitting sheet (4) which are spaced out from each other or are at least partly adjoined.

17. The fragrance-emitting item of claim 5, comprising either a single fragrance-emitting sheet (4) or several fragrance-emitting sheet (4) which are spaced out from each other or are at least partly adjoined.

18. The fragrance-emitting item of claim 2, comprising either a single fragrance-emitting sheet (4) or several fragrance-emitting sheet (4) which are spaced out from each other or are at least partly adjoined.

19. The fragrance-emitting item of claim 6, comprising either a single fragrance-emitting sheet (4) or several fragrance-emitting sheet (4) which are spaced out from each other or are at least partly adjoined.

20. The fragrance-emitting item of claim 7, comprising either a single fragrance-emitting sheet (4) or several fragrance-emitting sheet (4) which are spaced out from each other or are at least partly adjoined.
